SEQUENCE LISTING (6 sequences)

SEQ ID NO: 1 - DNA, 61 bp, synthetic construct
  source 1..61: mol_type = other DNA
gccggaagtcgtcactcttaagaagggacggggccccacgctgcgcacccgcgggtttgct

SEQ ID NO: 2 - DNA, 155 bp, synthetic construct
  source 1..155: mol_type = other DNA
gtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgct

SEQ ID NO: 3 - DNA, 303 bp, synthetic construct
  source 1..303: mol_type = other DNA
gtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgct

SEQ ID NO: 4 - DNA, 885 bp, synthetic construct
  source 1..885: mol_type = other DNA
atggccatgagcagcggcggcagtggcggcggcgtgcccgagcaggaggattctgtgctgttccggagaggaacaggccagagcgatgactccgatatctgggacgacacagcccttatcaaggcctacgacaaggccgtggccagctttaagcacgccctgaagaatggcgatatctgcgagacaagcggaaagcctaagaccacccctaaaagaaagcccgccaagaaaaacaagtcccagaaaaaaaacaccgccgctagcctgcagcagtggaaggtgggcgacaaatgcagcgccatctggtccgaggacggctgcatctaccctgctaccatcgccagcatcgacttcaagcgggaaacctgcgtggtggtctacacaggctatggcaatagggaggaacaaaatctctctgatctgctgtctcctatttgtgaagtggctaacaacatcgagcagaacgcccaggaaaatgagaacgaaagccaagtgtccaccgacgagagcgagaacagcagaagccctggaaacaagtctgacaacatcaagcccaagtctgccccttggaacagcttcctgccccctcctcctccaatgcctggccccagactgggccccggcaagcctggcctgaagttcaacggccctcctccaccccctcctcctccacctccccatctgctgagctgctggctgcctccttttcccagcggcccccctatcatccccccaccacctcctatctgtcccgacagcctggacgacgccgatgctctgggatccatgctgatcagctggtacatgtctggctaccacaccggctactacatgggcttccggcagaaccagaaggaaggaagatgcagccacagcctgaactga

SEQ ID NO: 5 - DNA, 2050 bp, synthetic construct
  source 1..2050: mol_type = other DNA
tcgaagctttataaaaacatacttttttttttacttttttttttttttctgagacacagcctcactctgtcgcccaggctggagtgcaggttttcatgtttatctgtgagatgtacctttggcacattactttcctgacatgagatttaaatttttttttttatcttgtgacaatttaacttttttgacacataaaaattgtacatatttatttgtttgagatggagtcgcactctgtcactcaggctggagtgcagtggcgtgatcttggctcactgcaacctccgcctcccgagttcaagtgattctcctggctcagcctcccaagcagctgtcattacaggcctgcaccaccacacccggctgattttgtatttttaggagaaacagggtttcaccatgttgggccaggctggtcttgaagtcctgacctcaagtgatccacccaccttggcctcccaaagtgctgggattataggcatgagccaccgtaccagacccctaaaaattgtatatatttaaggtgtaccatttgatgtttagatatacattgtgaaatgattacattccacatattacctctacagagttaccatttttgtacacttggtcaacatcatcccattctccccttcctccacagatatttcttgtatactatatagaagccaagggtattttgggggaagagctcaaagttcctttcgtggagttaaaaatatatatatactatgtacatataagccatttagcaaccctagatgcttaataaagaatactggaggcccggtgtggtggctcacacctgtaatcccagcactttgggaggccgaggcggtcggattacgaggtcaggagttcaagaccagcctggccaacatggtgaaaccccatctttactaaaaatacaaaaattagccgggtgtggtggtgggcgcctgtaatcccagctactcggggggctgaggcagaattgcttgaacctgggaggcagaggttgcagtgagctgagatcacgccactgcattccagcctgggtgacagagcaatattctgtcgcaaaaaaaaaaagaatactggaggctgggcgaggtggctcacacctgtaatcccagcattttgggatgccagaggcgggcggaatntcttgagctcaggagttcgagaccagcctacacaatatgctccaaacgccgcttntacaaaacatacagaaactacccgggtgtggtggcgnncccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgct

SEQ ID NO: 6 - DNA, 928 bp, synthetic construct
  source 1..928: mol_type = other DNA
ggatgccagaggcgggcggaatntcttgagctcaggagttcgagaccagcctacacaatatgctccaaacgccgcttntacaaaacatacagaaactacccgggtgtggtggcgnncccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgct

(19) United States
(12) Patent Application Publication
(10) Pub. No.: US 2025/0319208 A1
(43) Pub. Date: Oct. 16, 2025

(54) NEURON SPECIFIC PROMOTERS FOR AAV GENE TRANSFER

(71) Applicant: University of Massachusetts, Westborough, MA (US)

(72) Inventors: Jun Xie, Worcester, MA (US); Guangping Gao, Worcester, MA (US); Xiupeng Chen, Worcester, MA (US); Hong Ma, Worcester, MA (US)

(73) Assignee: University of Massachusetts, Westborough, MA (US)

(21) Appl. No.: 19/250,189

(22) Filed: Jun. 26, 2025

Related U.S. Application Data
(63) Continuation of application No. PCT/US2024/024748, filed on Apr. 16, 2024.
(60) Provisional application No. 63/496,440, filed on Apr. 17, 2023.

(51) Int. Cl. A61K 48/00; C12N 15/86

(52) U.S. Cl. CPC A61K 48/0058; C12N 15/86; C12N 2750/14143; C12N 2800/22

(57) ABSTRACT

Aspects of the disclosure relate to compositions and methods for delivery of certain gene products (e.g., proteins, nucleic acids, etc.) to a subject, e.g., to the central nervous system (CNS) of a subject. The disclosure is based, in part, on expression cassettes (e.g., isolated nucleic acids) comprising a nucleic acid sequence operably linked to a human SMN1 promoter or a variant of a human SMN1 promoter, for example variants that are truncated relative to native SMN1 promoters.

### RELATED APPLICATIONS

This application claims priority under 35 U.S.C. $119 (e) to U.S. Provisional Patent Application Ser. No. 63/496,440, filed Apr. 17, 2023, and entitled “NEURON SPECIFIC PROMOTERS FOR AAV GENE TRANSFER,” which is herein incorporated by reference in its entirety for all purposes.

### BACKGROUND OF INVENTION

Recombinant adeno-associated viral (rAAV) vectors are currently considered the leading platform for in vivo gene therapy. However, the packaging capacity of AAV is limited to <5 kb, which excludes larger therapeutic genes from conventional vector designs. Many efforts have been developed to overcome this limitation, such as split rAAVs and fragmented rAAV strategies.

### SUMMARY OF INVENTION

Aspects of the disclosure relate to compositions and methods for expressing heterologous gene products (e.g., proteins, such as therapeutic proteins, and functional RNAs such as interfering nucleic acids) in target cells of a subject. In some embodiments, the target cells are cells of the nervous system (e.g., CNS or PNS) of a subject. The disclosure is based, in part, on variants of SMN1 promoters that are beneficial for packaging and expressing larger transgenes. In some embodiments, the promoters are truncated SMN1 promoters (e.g., promoters having nucleotide sequences that are shortened relative to a native SMN1 promoter, for example a human SMN1 promoter, such as those set forth in SEQ ID NOs: 5 and 6).

Accordingly, in some aspects, the disclosure provides an isolated nucleic acid comprising a human SMN1 promoter variant comprising a nucleic acid sequence that is at least 60% identical to a nucleic acid sequence set forth in any one of SEQ ID NOs: 1-3 and does not comprise the sequence set forth in SEQ ID NO: 5 or 6.

In some embodiments, a human SMN1 promoter variant comprises a nucleic acid sequence set forth in SEQ ID NO: 1. In some embodiments, a human SMN1 promoter variant comprises a nucleic acid sequence set forth in SEQ ID NO: 2. In some embodiments, a human SMN1 promoter variant comprises a nucleic acid sequence set forth in SEQ ID NO: 3.

In some embodiments, an isolated nucleic acid further comprises a protein coding nucleic acid sequence operably linked to the promoter. In some embodiments, the protein coding nucleic acid encodes a therapeutic protein. In some embodiments, the therapeutic protein is a Survival Motor Neuron (SMN) protein.

In some embodiments, an isolated nucleic acid comprises an interfering nucleic acid sequence operably linked to the human SMN1 promoter. In some embodiments, the interfering nucleic acid is a dsRNA, SIRNA, shRNA, miRNA, artificial miRNA (ami-RNA), or RNA aptamer.

In some embodiments, an isolated nucleic acid further comprises a polyA region positioned 3′ relative to the nucleic acid sequence encoding the protein or the interfering nucleic acid.

In some embodiments, an isolated nucleic acid further comprises one or more miRNA binding sites.

In some embodiments, an isolated nucleic acid further comprises adeno-associated virus (AAV) inverted terminal repeats (TTRs).

In some aspects, the disclosure provides vector comprising an isolated nucleic acid as described herein.

In some aspects, the disclosure provides a recombinant AAV (rAAV) comprising an isolated nucleic acid as described herein: and at least one AAV capsid protein.

In some embodiments, at least one capsid protein has a serotype selected from an AAV2, AAV3, AAV4, AAV5, AAV6, AAV7, AAV8, AAVrh8, AAV9, AAV10, or AAVrh10 serotype. In some embodiments, at least one capsid protein is an AAV9 capsid protein.

In some embodiments, an rAAV is a self-complementary AAV (scAAV). In some embodiments, an rAAV is a self-complementary AAV9 (scAAV9).

In some embodiments, a protein coding nucleic acid sequence encodes a therapeutic protein. In some embodiments, a protein coding nucleic acid sequence encodes a Survival Motor Neuron (SMN) protein.

In some aspects, the disclosure provides a method of expressing a gene product in a subject, the method comprising administering an isolated nucleic acid or rAAV as described herein to the subject.

In some embodiments, a subject is a mammal. In some embodiments, a subject is a human.

In some embodiments, administration to a subject comprises injection. In some embodiments, injection comprises direct injection to the CNS of the subject or systemic injection.

In some embodiments, a gene product is a therapeutic protein encoded by an isolated nucleic acid or rAAV.

In some embodiments, administration of an isolated nucleic acid or rAAV results in expression of a gene product in CNS cells or peripheral nervous system (PNS) cells of a subject. In some embodiments. CNS cells comprise spinal cord neurons.

### BRIEF DESCRIPTION OF DRAWINGS

FIG. 1A-FIG. 1B show schematic representation of designed plasmids and vectors with AAV9 serotype. FIG. 1A shows plasmids with EGFP cDNA (enhanced green fluorescent protein complementary DNA) driven by three different versions of Human SMN1 (survival motor neuron 1) promoters, or driven with CB (chicken β-actin) promoter and CMV (cytomegalovirus) enhancer (CMVen/CB) as positive control. FIG. 1B shows packaged double-stranded AAV9 vectors containing different SMN1 and CB promoters.

FIG. 2A-FIG. 2B show GFP expression in vitro. FIG. 2A shows EGFP expression in HEK293 cells (immortalized human embryonic kidney cells). FIG. 2B shows EGFP expression in Neuro 2a cells (mouse neuroblasts). A 12-well plate was used to culture both cell lines, and 800 ng plasmids were added into each well with Lipofectamine 2000 Transfection Reagent. After 24 hours, images were captured with Leica DMi8, an inverted microscope for industrial applications. Scale bars are equal to 200 μm.

FIG. 3A-FIG. 3C show a Western blot analysis of EGFP expression after transfection. FIG. 3A shows EGFP expression in HEK293 cells. FIG. 3B shows EGFP expression in Neuro 2a cells. FIG. 3C shows quantification data for Western blot in FIG. 3A and FIG. 3B. There were three different repeats in each group. 12-well plates were used to culture both cell lines, 800 ng plasmids were added into each well with Lipofectamine 2000 Transfection Reagent. Cells were collected after 24 hours. Tub is short for Tubulin as the loading control.

FIG. 4A-FIG. 4C shows EGFP expression in central nerve system and periphery tissues at day 21 after intravenous injection at postnatal day 1 (P1). FIG. 4A shows EGFP expression in brain. FIG. 4B shows EGFP expression in the spinal cord, along with showing DAPI (4′,6-diamidino-2-phenylindole) and NeuN. FIG. 4C shows EGFP expression in the liver, heart, muscle, and pancreas. 155 hp and 303 hp promoter drove EGFP expression in the brain, spinal cord, liver, heart, and muscle. 61 bp-SMN1 promoter drove EGFP expression in the spinal cord, liver, and heart. None of these short promoters had activity in the pancreas. All mice were injected with a 4×10¹¹ vg/mouse at P1 with facial vein, and tissues were harvested around P21. 3 to 4 different animals were in each group. Muscle here is represented by the quadriceps. The scale bar in the brain images (FIG. 4A) is 50 microns, the scale bar in the spinal cord images (FIG. 4B) is 0.5 mm, and the scale bar in the images in FIG. 4C is 200 μm.

FIG. 5A-FIG. 5H shows a Western blot analysis for EGFP expression in mice. FIG. 5A shows EGFP expression in the spinal cord. FIG. 5B shows EGFP expression in the brain. FIG. 5C shows EGFP expression in the heart. FIG. 5D shows EGFP expression in the muscle. FIG. 5E-FIG. 5H shows the quantification of Western blots in FIG. 5A-FIG. 5D. All mice were injected with a 4×10¹¹ vg/mouse at P1 with facial vein, and tissues were harvested around P21. There were 3 to 4 different animals in each group. One-way ANOVA followed with Dunnett's multiple comparisons test. Muscle here is represented by the quadriceps; P-less stands for promoterless; NC stands for negative control (no injection); ne stands for no significance. *** stands for P<0.0001.

FIGS. 6A-6B show characterization of SMN1 promoters at different lengths in mice. FIG. 6A shows SMN1 promoters at different lengths and their activities in mouse brain and spinal cord. The 155-bp SMN1 promoter was trimmed from its 5′ or 3′ end to generate multiple EGFP reporter plasmids and packaged into AAV9 to evaluate their promoter activities in mice. Neonatal B6 mice were injected by facial vein et P1 at the dose 4×10¹¹ vector genomes (vg) per animal and tissues were harvested for analysis after 4 weeks of injection. The GFP expression levels are summarized at the right side. FIG. 6B shows GFP expression in mouse brain and spinal cord. N=3 in each group.

FIGS. 7A-7C shows evaluation of the bidirectional activity of 155 bp-SMN1 promoter in HEK 293 cells. FIG. 7A shows diagrams of AAV constructs. FIG. 7B shows EGFP expression in HEK 293 cells after 48 hours of plasmid transfection. FIG. 7C shows illuminance activity of Gaussia luciferase from the supernatant after 48 hours of transfection.

FIG. 8 shows evaluation of 155-bp SMN1 promoter in mice. An AAV plasmid harboring the 155-bp SMN1 promoter in the middle flanking with the mCherry gene in the upstream and EGFP gene in the downstream was packaged into AAV9. Facial vein administration to neonatal C57BL/6 mice at P1 with a dose of 4×10¹¹ vg/mouse. One month later, the animals were euthanized for analyzing the GFP and mCherry expression. N=3-5 in each group.

### DETAILED DESCRIPTION OF INVENTION

Aspects of the disclosure relate to compositions and methods for delivery of certain gene products (e.g., proteins, nucleic acids, etc.) to a subject, e.g., to the central nervous system (CNS) of a subject. The disclosure is based, in part, on expression cassettes (e.g., isolated nucleic acids) comprising a nucleic acid sequence operably linked to a human SMN1 promoter or a variant of a human SMN1 promoter, for example variants that are truncated relative to a native SMN1 promoter, for example a human SMN1 promoter (e.g., promoters comprising the sequence set forth in SEQ ID NO: 5 or 6). In some embodiments, a human SMN1 promoter is capable of driving physiological expression level of SMN in certain cells of a subject, for example CNS cells (e.g., spinal cord neurons) of a human subject. The human SMN1 promoter variants may be used as a portion of an isolated nucleic acid (e.g., DNA, plasmid vector, rAAV vector, etc.) or as a portion of a viral particle, for example a recombinant adeno-associated virus (rAAV) or recombinant lentiviral particle.

Aspects of the disclosure also provide methods for delivering a transgene (e.g., a gene product, for example a therapeutic protein and/or inhibitory nucleic acid) to a target tissue of a subject, such as CNS tissue. The disclosure is based, in part, on human SMN1 promoter variants that are truncated (e.g., shortened) relative to a native human SMN1 promoter and allow for efficient transgene expression in certain cells (e.g., CNS cells of a subject) while reducing the promoter size. In some embodiments, reduced promoter size is useful for incorporating such promoters into vectors having limited transgene capacity, for example rAAV vectors. In some embodiments, delivery of a transgene (e.g., SMN1) to CNS cells of a subject is useful for treating central nervous system (CNS) diseases.

### Human SMN1 Promoters

Aspects of the disclosure relate to nucleic acids and vectors, for example viral vectors, that comprise a human SMN1 promoter or a variant of a human SMN 1 promoter (e.g., as described in any one of SEQ ID NOs: 1-3). A “promoter” refers to a DNA sequence recognized by the synthetic machinery of the cell, or introduced synthetic machinery, required to initiate the specific transcription of a gene. The phrases “operatively linked.” “operatively positioned.” “under control” or “under transcriptional control” means that the promoter is in the correct location and orientation in relation to the nucleic acid to control RNA polymerase initiation and expression of the gene.

In some embodiments, the promoter (e.g., human SMN1 promoter) imparts tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.,) are well known in the art.

In some embodiments, the human SMN1 promoter preferentially drives expression of a gene product encoded by a nucleic acid in certain tissues. In some embodiments, the disclosure provides a nucleic acid comprising a tissue-specific human SMN1 promoter (e.g., a human SMN1 promoter or a human SMN1 promoter variant operably linked to a nucleic acid sequence (e.g., a transgene encoding a protein, such as a therapeutic protein, e.g., SMN1). As used herein, “tissue-specific promoter” refers to a promoter that preferentially regulates (e.g., drives or up-regulates) gene expression in a particular cell type relative to other cell types. A cell-type-specific promoter can be specific for any cell type, such as central nervous system (CNS) cells, liver cells (e.g., hepatocytes), heart cells, muscle cells, etc. In some embodiments, a tissue-specific promoter is a CNS-tissue-specific, PNS-tissue-specific, or cell-specific promoter. In some embodiments, the human SMN1 promoter (or human SMN1 promoter variant) is tissue-specific to, e.g., kidney cells, neuroblasts, spinal cord neurons, brain cells, liver cells, heart cells, and/or muscle cells.

Aspects of the disclosure relate to isolated nucleic acids and rAAV vectors comprising a nucleic acid sequence encoding a gene product operably linked to its native promoter. In some embodiments a native promoter comprises a human SMN1 promoter, or a variant thereof (e.g., as described in any one of SEQ ID NOs: 1-3). In some embodiments, a human SMN1 promoter variant comprises a nucleic acid sequence that has at least 20%, at least 30%, at least 40%, at least 50%, at least 60%, at least 70%, at least 80%, at least 90%, at least 91%, at least 92%, at least 93%, at least 945%, at least 95%, at least 969%, at least 97%, at least 98%, at least 99%, or 100% sequence identity with the nucleic acid sequence set forth in one of SEQ ID NOs: 1-3. A native promoter may be preferred when it is desired that expression of the transgene should mimic the native expression (e.g., express physiological levels of a gene product, for example a therapeutic protein such as SMN, expression in the appropriate cell types). The native promoter may be used when expression of the transgene must be regulated temporally or developmentally, or in a tissue-specific manner, or in response to specific transcriptional stimuli. Without wishing to be bound by any theory, use of a human SMN1 promoter in isolated nucleic acids and rAAV vectors described herein regulates expression of gene products from the vectors, and reduces toxicity, for example cytotoxicity or hepatotoxicity, in a subject relative to expression of the gene products from isolated nucleic acids and rAAV vectors comprising other promoters, for example CMV promoter, chicken-beta actin (CBA) promoter, CB6 promoter, etc. In a further embodiment, other native expression control elements, such as enhancer elements, polyadenylation sites, and/or Kozak consensus sequences may also be used to mimic the native expression.

In some embodiments, the nucleic acid sequence encoding SMN is operably linked to a human SMN1 promoter. In certain embodiments, a human SMN1 promoter is configured to drive physiological expression level of a gene product (e.g., a therapeutic protein or an interfering RNA). In some embodiments, the human SMN1 promoter comprises a sequence that is set forth in one of SEQ ID NOs: 1-3. In some embodiments, the human SMN1 promoter consists of a sequence that is set forth in one of SEQ ID NOs: 1-3.

In some embodiments, the nucleic acid comprises the sequence set forth in one of SEQ ID NOs: 1.3. In some embodiments, the nucleic acid has a sequence having at least 10% sequence identity, at least 20% sequence identity, at least 30% sequence identity, at least 40% sequence identity, at least 50% sequence identity, at least 55% sequence identity, at least 60% sequence identity, at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, at least 99% sequence identity, or 1009% sequence identity with one of SEQ ID NOs: 1-3. In certain embodiments, the nucleic acid has a sequence having 100% sequence identity with one of SEQ ID NOs: 1-3. In certain embodiments, a human SMN1 promoter variant has unexpectedly beneficial performance in spinal cord neurons, neurons and/or muscle cells.

In some embodiments, the nucleic acid has a human SMN1 promoter variant sequence that is truncated relative to SEQ ID NO: 5 or 6 by at least 1, 2, 3, 4, 5, 6, 7, 8, 9, 10, or more nucleotides. In some embodiments, the nucleic acid has a human SMN1 promoter variant sequence that is truncated relative to SEQ ID NO: S or 6 by at least 20, 50, 75, 100, 140, 148, 200, 240, 250, 300, 350, 400, 450, 500, or more nucleotides.

In some embodiments, the human SMN1 promoter has a length of at least 61 base pairs, at least 65 base pairs, at least 70 base pairs, at least 75 base pairs, at least 80 base pairs, at least 85 base pairs, or at least 90 base pairs. In some embodiments, the human SMN1 promoter has a length of at most 303 base pairs, at most 300 base pairs, at most 250 base pairs, at most 200 base pairs, at most 155 base pairs, at most 150 base pairs, at most 145 base pairs, at most 140 base pairs, at most 135 base pairs, at most 130 base pairs, at most 125 base pairs, at most 120 base pairs, at most 115 base pairs, at most 110 base pairs, at most 105 base pairs, at most 100 base pairs, at most 95 base pairs. Combinations of the above-referenced ranges are also possible (e.g., at least 61 base pairs and at most 303 base pairs, at least 61 base pairs and at most 155 base pairs). Other ranges are also possible.

The terms “percent identity.” sequence identity. “% identity,” % sequence identity.” and % identical,” as they may be interchangeably used herein, refer to a quantitative measurement of the similarity between two sequences (e.g., nucleic acid or amino acid).

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 909%, at least 959%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk. A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press. 1987; Computer Analysis of Sequence Data, Part I. Griffin, A. M., and Griffin, H. G., eds., Humana Press, New Jersey. 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman. D., SIAM J Applied Math., 48:1073 (1988); incorporated herein by reference. Techniques for determining identity are codified in publicly available computer programs. Exemplary computer software to determine homology between two sequences include, but are not limited to, OCG program package, Devereux. J., et al., Nucleic Acids Research, 12 (1), 387 (1984)). BLASTP, BLASTN, and FASTA Atschul, S. F. et al., J. Molec. Biol., 215, 403 (1990)).

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 759%, at least 76%, at least 77%, at least 78%, at least 79%, at least 80%, at least 81%, at least 826%, at least 83%, at least 84%, at least 85%, at least 86%, at least 87%, at least 889%, at least 89%, at least. 909%, at least 919%, at least 92%, at least 939%, at least 94%, at least 95%, at least 95.5%, at least 96%, at least 96.5%, at least 97%, at least 97.5% at least 98%, at least 98.59%, at least 99%, at least 99.5%, at least 99.69%, at least 99.79%, at least 99.89%, at least 99.9% identity) and all increments thereof (e.g., tenths of a percent (e.g., 0.1%), hundredths of a percent (e.g., 0.01%), etc.).

### Isolated Nucleic Acids

Aspects of the disclosure relate to nucleic acids, for example isolated nucleic acids. A “nucleic acid” sequence refers to a DNA or RNA sequence. In some embodiments, proteins and nucleic acids of the disclosure are isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See. e.g., texts such as Sambrook ci al., “Molecular Cloning. A Laboratory Manual”, 2d ed., Cold Spring Harbor Laboratory, New York (1989); and K. Fisher et al., J Virol, 70:520 532 (1996). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV TTRs are AAV2 TTRs. In some embodiments, at least one AAV ITR is a truncated AAV ITR (e.g., a mutant ITR, also referred to as an mTR), for example a ΔITR as described, for example by McCarty (2008) Molecular Therapy 16 (10): 1648-1656.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence; sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein of polypeptide. Similarly, two or more coding regions are operably linked when they are linked in such a way that their transcription from a common promoter results in the expression of two or more proteins having been translated in frame. In some embodiments, operably linked coding sequences yield a fusion protein.

A region comprising a transgene (e.g., a transgene encoding a gene product, for example SMN protein, etc.) may be positioned at any suitable location of the isolated nucleic acid that will enable expression of the at least one transgene, the selectable marker protein, or reporter protein.

For nucleic acids encoding proteins, a polyadenylation sequence generally is inserted following the transgene sequences and before the 3′ AAV ITR sequence. A rAAV construct useful in the disclosure may also contain an intron, desirably located between the promoter/enhancer sequence and the transgene. One possible intron sequence is derived from SV-40, and is referred to as the SV-40 T intron sequence. In some embodiments, an intron is a non-native intron or synthetic intron (e.g., a MBL intron). Another vector element that may be used is an internal ribosome entry site (IRES). An IRES sequence is used to produce more than one polypeptide from a single gene transcript. An IRES sequence would be used to produce a protein that contains more than one polypeptide chains. Selection of these and other common vector elements are conventional, and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4:928-933; Mattion, N M et al., J Virology, November 1996; p. 8124-8127; Furler, S et al., Gene Therapy, 2001; 8:864-873; and Halpin, C et al., The Plant Journal, 1999; 4:453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8:864-873; and Halpin, C et al., The Plant Journal, 1999; 4:453-459; de Felipe, P et al., Gene Therapy, 1999; 6: 198-208: de Felipe, P et al., Human Gene Therapy, 2000; 11:1921-1931.; and Klump, H et al., Gene Therapy, 2001; 8:811-817).

In some embodiments, one or more bindings sites for one or more of miRNAs are incorporated in a transgene of a rAAV vector, to inhibit the expression of the transgene in one or more tissues of an subject harboring the transgene. The skilled artisan will appreciate that binding sites may be selected to control the expression of a transgene in a tissue specific manner. For example, binding sites for the liver-specific miR-122 may be incorporated into a transgene to inhibit expression of that transgene in the liver. The target sites in the mRNA may be in the 5′ UTK, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISC's and provide highly efficient inhibition of expression. The target site sequence may comprise a total of 5-100, 10-60, or more nucleotides. The target site sequence may comprise at least 5 nucleotides of the sequence of a target gene binding site.

### Gene Products

Aspects of the disclosure relate to isolated nucleic acids comprising a human SMN1 promoter or human SMN1 promoter variant operably linked to a nucleic acid sequence encoding a gene product. A gene product may be a peptide, protein, nucleic acid, or a combination thereof. In some embodiments, the nucleic acid gene product is a therapeutic functional RNA (e.g., an interfering RNA, such as dsRNA, siRNA, miRNA, artificial miRNA (ami-RNA), RNA aptamer, etc.). In some embodiments, a nucleic acid comprising a human SMN1 promoter or human SMN1 promoter variant operably linked to a nucleic acid sequence encoding a gene product is referred to as a transgene. In some embodiments, a transgene further comprises one or more additional regulatory sequences, such as an enhancer, polyA region, etc.

For example, one type of transgene sequence includes a reporter sequence, which upon expression produces a detectable signal. In another example, the transgene encodes a therapeutic protein or therapeutic functional RNA. In another example, the transgene encodes a transmembrane protein. In another example, the transgene encodes a secreted protein. In another example, the transgene encodes a protein or functional RNA that is intended to be used for research purposes, e.g., to create a somatic transgenic animal model harboring the transgene. e.g., to study the function of the transgene product. In another example, the transgene encodes a protein or functional RNA that is intended to be used to create an animal model of disease. Appropriate transgene coding sequences will be apparent to the skilled artisan.

Reporter sequences that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, Ovalbumin (OVA) and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of an rAAV.

In some aspects, the disclosure provides vectors (e.g., TAAV vectors, lentiviral (LV) vectors, adenoviral vectors, plasmids, cosmids, etc.) for use in methods of preventing of treating one or more genetic deficiencies or dysfunctions in a mammal, such as for example, a polypeptide deficiency or polypeptide excess in a mammal, and particularly for treating or reducing the severity or extent of deficiency in a human manifesting one or more of the disorders linked to a deficiency in such polypeptides in cells and tissues. The method involves administration of a vector that encodes one or more therapeutic peptides, polypeptides, siRNAs, microRNAs, antisense nucleotides, die, in a pharmaceutically-acceptable carrier to the subject in an amount and for a period of time sufficient to treat the deficiency or disorder in the subject suffering from such a disorder.

Thus, the disclosure embraces the delivery of vectors encoding one or more peptides, polypeptides, or proteins, which are useful for the treatment or prevention of disease states in a mammalian subject. Exemplary therapeutic proteins include one or more polypeptides selected from the group consisting of growth factors, interleukins, interferons, anti-apoptosis factors, cytokines, anti-diabetic factors, anti-apoptosis agents, coagulation factors, anti-tumor factors. Other non-limiting examples of therapeutic proteins include BDNF, CNTF, CSF, EGF, FGF, G-SCF, GM-CSF, gonadotropin, IFN, IFG-1, M-CSF, NGF, PDGF, PEDF, TOF, VEGF, TGF-B2, TNF, prolactin, somatotropin, XIAP1, IL-1, IL-2, IL-3, IL-4, IL-5, IL-6, IL-7, IL-8, IL-9, IL-10, IL-10 (187A), viral IL-10, IL-11, IL-12, IL-13, IL-14, IL-15, IL-16 IL-17, and IL-18. In some

In some embodiments, therapeutic proteins include any polypeptide that is suitable for the purpose of delivering the vector and/or treating or preventing a disease.

The vectors disclosed herein may comprise a transgene to be transferred to a subject to treat a disease associated with reduced expression, lack of expression or dysfunction of the native gene. Exemplary genes and associated disease states include, but are not limited to: glucose-6-phosphatase, associated with glycogen storage deficiency type 1A; phosphoenolpyruvate-carboxykinase, associated with Pepck deficiency; galactose-1 phosphate uridyl transferase, associated with galactosemia; phenylalanine hydroxylase, associated with phenylketonuria; branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia: medium chain acyl CoA dehydrogenase, associated with medium chain acetyl CoA deficiency; omithine transcarbamylase, associated with omithine transcarbamylase deficiency; argininesuccinic acid synthetase, associated with citrullinemia; low density lipoprotein receptor protein, associated with familial hypercholesterolemia: UDP-glucoaronosyltransferase, associated with Crigler-Najjar disease; adenosine deaminase, associated with severe combined immunodeficiency disease: hypoxanthine guanine phosphoribosyl transferase, associated with Gout and Lesch-Nyan syndrome; biotinidase, associated with biotinidase deficiency; beta-glucocerebrosidase, associated with Gaucher disease: beta-glucuronidase, associated with Sly syndrome; peroxisome membrane protein 70 kDa, associated with Zellweger syndrome; porphobilinogen deaminase, associated with acute intermittent porphyria; alpha-1 antitrypsin for treatment of alpha-1 antitrypsin deficiency (emphysema): erythropoietin for treatment of anemia due to thalassemia or to renal failure; vascular endothelial growth factor, angiopcictin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergie receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and atrophin or miniutrophin for the treatment of muscular dystrophies; and, insulin for the treatment of diabetes.

The following are further non-limiting examples of gene products (e.g., proteins) that may be encoded by transgenes of the vectors disclosed herein to treat a disease associated with reduced expression, lack of expression or dysfunction of the native gene: a-galactosidase, acid-glucosidase, adiopokines, adiponectin, alglucosidase alfa, anti-thrombin, ApoAV, ApoCII, apolipoprotein A-1 (APOA1), arylsulfatase A, arylsulfatase B, ATP-binding cassette transporter A1 (ABCA1), ABCD1, CCR5 receptor, erythropoietin, Factor VIII, Factor VII, Factor IX, Factor V, fetal hemoglobin, beta-globin, GPI-anchored HDL-binding protein (GPI-HBP) I, growth hormone, hepatocyte growth factor, imiglucerase, lecithin-cholesterol acyltransferase (LCAT), leptin, LDL receptor, lipase maturation factor (LMF) 1, lipoprotein lipase, lysozyme, nicotinamide dinucleotide phosphate (NADPH) oxidase, Rab escort protein-1 (REP-1), retinal degeneration slow (RDS), retinal pigment epithelium-specific 65 (RPE65), rhodopsin, T cell receptor alpha or beta chains, thrombopoeitin, tyrosine hydroxylase, VEGF, von heldebrant factor, von willebrand factor, and X-linked inhibitor of apoptosis (XIAP).

In some embodiments, the vectors may comprise a gene encoding an antigen-binding protein, such as an immunoglobulin heavy chain or light chain or fragment thereof, e.g., that may be used for therapeutic purposes. In some embodiments, the protein is a single chain Fv fragment or Fv-Pc fragment. Accordingly, in some embodiments, the vector can be used to infect cells are of target tissue (e.g., muscle tissue) to engineer cells of the tissue to express an antigen-binding protein, such as an antibody or fragment thereof. In some embodiments, to generate vectors that express the antibodies or antigen binding fragments, cDNAs engineered to express such proteins will be subcloned into an appropriate plasmid backbone and packaged into a viral vector, for example an rAAV.

The vector of the disclosure can be used to restore the expression of genes that are reduced in expression, silenced, or otherwise dysfunctional in a subject (e.g., a tumor suppressor that has been silenced in a subject having cancer). The vectors of the disclosure can also be used to knockdown the expression of genes that are aberrantly expressed in a subject (e.g., an oncogene that is expressed in a subject having cancer).

The skilled artisan will also realize that in the case of transgenes encoding proteins or polypeptides, that mutations that results in conservative amino acid substitutions may be made in a transgene to provide functionally equivalent variants, or homologs of a protein or polypeptide. In some aspects, the disclosure embraces sequence alterations that result in conservative amino acid substitution of a transgene. In some embodiments, the transgene comprises a gene having a dominant negative mutation. For example, a transgene may express a mutant protein that interacts with the same elements as a wild-type protein, and thereby blocks some aspect of the function of the wild-type protein.

In some embodiments, the cloning capacity of the recombinant RNA vector may be limited and a desired coding sequence may require the complete replacement of the virus's 4.8 kilobase genome. Large genes may, therefore, not be suitable for use in a standard recombinant AAV vector, in some cases. The human SMN1 promoter variants in the instant disclosure advantageously overcome a limited coding capacity due to their small number of nucleotides (e.g., less than or equal to 303 bp).

It should be appreciated that in cases where a transgene encodes more than one gene product (e.g., a therapeutic protein, and another protein or interfering nucleic acid), each gene product may be positioned in any suitable location within the transgene. For example, a nucleic acid encoding a first polypeptide may be positioned in an intron of the transgene and a nucleic acid sequence encoding a second polypeptide may be positioned in another untranslated region (e.g., between the last codon of a protein coding sequence and the first base of the poly-A signal of the transgene).

Aspects of the disclosure relate to a nucleic acid comprising a nucleic acid sequence encoding an SMN protein, e.g., a codon optimized nucleic acid sequence encoding an SMN protein. Without wishing to be bound by any particular theory, methods and compositions described herein identify and/or correct metabolic imbalances in the CNS of a subject.

In some embodiments, the nucleic acid sequence encoding an SMN protein comprises SEQ ID NO: 4. In some embodiments, the nucleic acid sequence encoding an SMN protein has a sequence having at least 10% sequence identity, at least 20% sequence identity, at least 30% sequence identity, at least 40% sequence identity, at least 50% sequence identity, at least 55% sequence identity, at least 60% sequence identity, at least 70% sequence identity, at least 75% sequence identity, at least 80% sequence identity, at least 85% sequence identity, at least 90% sequence identity, at least 95% sequence identity, at least 96% sequence identity, at least 976% sequence identity, at least 98% sequence identity, at least 99% sequence identity, or 100% sequence identity with SEQ ID NO: 4. In certain embodiments, the nucleic acid sequence encoding an SMN protein has a sequence having 100% sequence identity with SEQ ID NO: 4. In some embodiments, the nucleic acid encoding an SMN protein has a sequence that is truncated relative to SEQ ID NO: 4 by at least 1 nucleotide and at most 5 nucleotides, at least 2 nucleotides and at most 10) nucleotides, at least 5 nucleotides and at most 20 nucleotides, up to 50% truncation, up to 60% truncation, up to 70% truncation, up to 80% truncation, or up to 90% truncation.

In some aspects, the disclosure relates to isolated nucleic acids comprising a transgene encoding one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in a 3′ untranslated region (3′ UTR) of a transgene, for example between the last codon of a nucleic acid sequence encoding one or more complement control proteins as described herein, and a poly A sequence.

In some embodiments, transgene expression causes overexpression of the transgene in the liver, resulting in liver toxicity (see. e.g., Hinderer et al., Severe Toxicity in Nonhuman Primates and Piglets Following High-Dose Intravenous Administration of an Adeno-Associated Virus Vector Expressing Human SMN, Volume: 29 Issue 3, 285-298: Mar. 1, 2018) In some embodiments, in order to reduce liver toxicity, the AAV vector comprises a transgene comprising one or more (e.g., 1, 2, 3, 4, 5, or more) miRNA binding sites that de-target expression of a transgene from liver cells. For example, in some embodiments, a transgene comprises one or more miR-122 binding sites. In some embodiments, the rAAV vector described herein comprises one or more miR-122 binding sites.

In some embodiments, a transgene comprises one or more (e.g., 1, 2, 3, 4, 5, or more) miRNA binding sites that de-target expression of a transgene from immune cells (e.g., antigen presenting cells (APCs), such as macrophages, dendrites, etc.). Incorporation of miRNA binding sites for immune-associated miRNAs may de-target transgene (e.g., one or more inhibitory nucleic acids) expression from antigen presenting cells and thus reduce or eliminate immune responses (cellular and/or humoral) produced in the subject against products of the transgene, for example as described in US 2018/0066279, the entire contents of which are incorporated herein by reference.

As used herein an “immune-associated miRNA” is an miRNA preferentially expressed in a cell of the immune system, such as an antigen presenting cell (APC). In some embodiments, an immune-associated miRNA is an miRNA expressed in immune cells that exhibits at least a 2-fold, 3-told, 4-told, 5-fold, 6-told, 7-told, 8-told, 9-fold, 10-fold higher level of expression in an immune cell compared with a non-immune cell (e.g., a control cell, such as a HeLa cell. HEK293 cell, mesenchymal cell, etc.). In some embodiments, the cell of the immune system (immune cell) in which the immune-associated miRNA is expressed is a B cell, T cell, Killer T cell, Helper T cell, γδ T cell, dendritic cell, macrophage, monocyte, vascular endothelial cell, or other immune cell. In some embodiments, the cell of the immune system is a B cell expressing one or more of the following markers: B220, BLAST-2 (EBVCS), Bu-1, CD19, CD20 (L26), CD22, CD24. CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2, CD1a, CD1d, CD11b (Mac-1), CD134 (OX40), CD150, CD2, CD25 (interleukin 2 receptor alpha), CD3, CD38, CD4, CD45RO, CD5, CD7, CD72, CD5, CRTAM, FOXP3, FT2, CPCA, HLA-DR, HML-1. HT23A, Leu-22, Ly-2, Ly-m22, MICG, MRC OX 8, MRC OX-22, OX40, PD-1 (Programmed death-1), RT6, TCR (T cell receptor). Thy-1 (CD90), and TSA-2 (Thymic shared Ag-2). In some embodiments, the immune-associated miRNA is selected from: miR-15a, miR-16-1, miR-17, miR-18a, miR-19a, miR·19b·1, miR-20a, miR-31, miR-29a/b/c, miR-30b, miR-31, miR·34a, miR-92a-1, miR-106a, miR-125a/b, miR-142-3p, miR-146a, miR-150, miR-155, miR-181a, miR-223 and miR-424, miR-221, miR-222, let-7i, miR-148, and miR-152. In some embodiments, a transgene described herein comprises one or more binding sites for miR-142.

Recombinant Adeno-Associated Viruses (rAAVs)

In some embodiments, an isolated nucleic acid as described herein is flanked by adeno-associated virus inverted terminal repeats (AAV ITRs).

The present disclosure provides a recombinant AAV (rAAV) comprising: (i) an isolated nucleic acid described herein and (ii) at least one AAV capsid protein. In some embodiments, the at least one capsid protein has a serotype selected from an AAV2, AAV3, AAV4, AAV5, AAV6, AAV7, AAV8, AAVrh8, AAV9, AAV10, or AAVrh10 serotype. In some embodiments, the at least one capsid protein is an AAV9 capsid protein. In some embodiments, the rAAV is a self-complementary AAV (scAAV). In some embodiments, the rAAV is a self-complementary AAV9 (scAAV9).

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (AAVs) preferably have tissue-specific targeting capabilities, such that a transgene of the rAAV will be delivered specifically to one or more predetermined tissue(s) (e.g., muscle tissues, ocular tissues, neurons, etc.). The AAV capsid is an important element in determining these tissue-specific targeting capabilities (e.g., tissue tropism). Thus, an rAAV having a capsid appropriate for the tissue being targeted can be selected.

In some embodiments, rAAVs of the disclosure comprise a nucleotide sequence as set forth in one of SEQ ID NOs: 1-3, and/or SEQ ID NO: 4. In some embodiments, rAAVs of the disclosure comprise a nucleotide sequence that is 99% identical, 95% identical. 90% identical. 85% identical, 80% identical, 75% identical, 70% identical, 65% identical, 60% identical. 55% identical, or 50% identical to a nucleotide sequence as set forth in one of SEQ ID NOs: 1-3, and/or SEQ ID NO: 4.

In some aspects, the present disclosure provides a recombinant adeno-associated virus (rAAV) comprising: (a) a self-complementary rAAV genome comprising: (i) a 5′ ITR; (ii) a human SMN1 promoter comprising the nucleotide sequence of one of SEQ ID NOs: 1-3; (iii) a codon optimized nucleic acid sequence encoding SMN as set forth in SEQ ID NO: 4; (iv) a poly A tail; and (v) a 3′ ITR; and (b) a AAV9 capsid protein. In some embodiments, the poly A tail is a rabbit globin poly A or a BGH poly A tail. In some embodiments, the rAAV farther comprises one or more miR-122 binding sites.

Methods for obtaining recombinant AAVs having a desired capsid protein are well known in the art. (See, for example, US 2003/0138772), the contents of which are incorporated herein by reference in their entirety) Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV, AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein has a tropism for central nervous system (CNS) tissues. In some embodiments, an AAV capsid protein targets neuronal cell types, astrocytes, oligodendrocytes, glial cells, etc. In some embodiments, an AAV capsid protein is of an AAV serotype selected from the group consisting of AAV1, AAV2, AAV3, AAV4, AAV5, AAV6, AAV7, AAV8, AAVrh8, AAV9, AAV10, AAVrh10, AAV.PHP-eB, AAVrh39, AAVrh43, and variants of any of the foregoing. In some embodiments, the rAAV comprises an AAV9 capsid protein.

In some embodiments, an rAAV vector or rAAV particle comprises a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR). Without wishing to be bound by any particular theory, a rAAV vector comprising an ITR lacking a functional TRS produces a self-complementary rAAV vector (scAAV or scrAAV vector), for example as described by McCarthy (2008) Molecular Therapy 16 (10): 1648-1656.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain ene or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid that comprises a codon-optimized coding sequence encoding a transgene (e.g., a transgene encoding SMN). A “host cell” refers to any cell that harbors, or is capable of harboring, a substance of interest. Often a host cell is a mammalian cell. In some embodiments, a host cell is a neuron. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a central nervous system cell, for example a neuron or a glial cell.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See. e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, etc. Thus, the term includes cloning and expression vehicles, as well as viral vectors. In some embodiments a vector comprises a baculovirus vector, which are useful for producing viral particles in certain insect cells (e.g., SF9 cells). In some embodiments, useful vectors are contemplated to be those vectors in which the nucleic acid segment to be transcribed is positioned under the transcriptional control of a promoter.

### Delivery of a Transgene to Tissue

Aspects of the disclosure relate to methods for delivering (e.g., expressing) a gene product in a target cell or tissue (e.g., a target cell or tissue of a subject). The disclosure is based, in part, on human SMN1 promoter variants that enhance expression of gene products in certain tissues, for example CNS tissues and PNS tissues. In some embodiments, the promoters described herein enhance gene product expression in certain cell types, for example spinal cord neurons and/or heart cells. In some embodiments, the methods of delivery comprise administering a nucleic acid or an rAAV described herein to the subject.

In some embodiments, delivery of a transgene comprising a human SMN1 promoter to a target tissue, e.g., CNS tissue, results in comparable expression of the gene product of the transgene relative to a transgene expressed from a different, longer promoter (e.g., chicken β-actin (CB) promoter, etc.) In some embodiments, expression of the gene product is comparable (e.g. within 50%, within 40%, within 30%, within 20%, within 10% as measured by Western Blot) relative to expression of a gene product expressed under the control of a longer promoter (e.g., 10% longer, 20% longer, 30% longer, 40% longer. 50% longer. 100% longer, 200% longer, or greater, as measured by number of nucleotides) (e.g., a CB promoter, etc.). Techniques for measurement of gene product expression levels are known, for example Western blotting, qPCR, next-generation sequencing, relative fluorescence, etc.

The isolated nucleic acids, rAAVs, and compositions of the disclosure may be delivered to a subject in compositions according to any appropriate methods known in the art. For example, an rAAV, preferably suspended in a physiologically compatible carrier (e.g., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human. In some embodiments, a subject is human. In some embodiments, the subject is a mammal. In some embodiments, the mammal is a human. In some embodiments, the subject has, is suspected of having, or is at risk of having, a neurological disorder. In some embodiments, the subject has, is suspected of having, or is at risk of having, a disease associated with the CNS.

In some embodiments, the administration comprises systemic administration. In some embodiments, the systemic administration comprises intravenous injection.

Delivery of the rAAVs may be by, for example intramuscular injection or infusion into the muscle tissue or cells of a subject. As used herein. “muscle tissues” refers to any tissue derived from or contained in skeletal muscle, smooth muscle, or cardiac muscle of a subject. Non-limiting examples of muscle tissues include skeletal muscle, smooth muscle, cardiac muscle, myocytes, sarcomeres, myofibrils, etc.

Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue.

Aspects of the instant disclosure relate to compositions comprising a nucleic acid encoding a transgene and a pharmaceutically acceptable carrier.

The compositions of the disclosure may comprise an isolated nucleic acid or vector alone, or in combination with one or more other components (e.g., a second isolated nucleic acid or vector encoding one or more different transgenes). In some embodiments, a composition comprises 1, 2, 3, 4, 5, 6, 7, 8, 9, 10, or more different isolated nucleic acids or vectors each having one or more different transgenes.

Suitable carriers may be readily selected by one of skill in the art in view of the indication for which the isolated nucleic acid or vector is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the disclosure.

Optionally, the compositions of the disclosure may contain, in addition to the isolated nucleic acid or vector, and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The isolated nucleic acids or vectors are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), intraocular injection, subretinal injection, oral, inhalation (including intranasal and intratracheal delivery), intravenous, intramuscular, subcutaneous, intradermal, intratumoral, and other parental routes of administration. Routes of administration may be combined, if desired.

The dose of isolated nucleic acid or vector (e.g., rAAV virions) required to achieve a particular “therapeutic effect.” e.g., units of dose in genome copies/per kilogram of body weight (GC/kg), or mass/volume units of dose, will vary based on several factors including, but not limited to the route of rAAV virion administration, the level of gene or RNA expression required to achieve a therapeutic effect, the specific disease of disorder being treated, and the stability of the gene or RNA product. For example, one of skill in the art can readily determine a rAAV virion dose range to treat a patient having a particular disease or disorder based on the aforementioned factors, as well as other factors that are well known in the art. In some embodiments, an rAAV as described herein is administered to a subject in a dose ranging between about 1 ml to about 100 ml of solution containing from about 10⁹ to 10¹⁶ genome copies. In some cases, a dosage between about 10¹¹ to 10¹³ rAAV genome copies is administered.

An effective amount of an rAAV is an amount sufficient to target infect an animal, target a desired tissue. In some embodiments, an effective amount of an rAAV is administered to the subject during a pre-symptomatic stage of degenerative disease. In some embodiments, a subject is administered an rAAV or composition after exhibiting one or more signs or symptoms of degenerative disease.

An effective amount of an rAAV may also depend on the mode of administration. For example, targeting a muscle tissue (e.g., muscle cells) by intramuscular administration or subcutaneous injection may require different (e.g., higher or lower) doses, in some cases, than targeting muscle tissue by another method (e.g., systemic administration, topical administration, etc.). In some embodiments, intramuscular injection (IM) of rAAV having certain serotypes (e.g., AAV2, AAV6, AAV9, etc.) mediates efficient transduction of muscle cells. Thus, in some embodiments, the injection is intramuscular injection (IM). In some embodiments, the injection is systemic administration (e.g., intravenous injection). In some cases, multiple doses of a rAAV are administered. In some embodiments, the administration is systemic administration. In some embodiments, the systemic administration comprises intravenous administration. In some embodiments, the administration is local administration to the central nervous system, for example by intracerebral injection, intrathecal injection, intracranial injection, etc.

In some embodiments, rAAV compositions are formulated to reduce aggregation of AAV particles in the composition, particularly where high rAAV concentrations are present (e.g., ˜10¹³ GC/mL or more). Methods for reducing aggregation of rAAVs are well known in the art and include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either intraocularly, subretinally, subcutaneously, intraopancreatically, intranasally, parenterally, intravenously, intramuscularly, intrathecally, orally, intraperitoneally, or by inhalation. In some embodiments, the administration modalities as described in U S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringeability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 am to 4 am. Sonication of ML Vs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708) microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Therapeutic Methods

Aspects of the disclosure relate to methods for preventing of treating certain neurological and/or neuromuscular diseases, for example Spinal Muscular Atrophy (SMA). In some embodiments, the method comprises administering a nucleic acid or an rAAV described herein to a subject. In some embodiments, the rAAV vector or nucleic acid comprises a human SMN1 promoter, for example as set forth in one of SEQ ID NOs; 1-3. The disclosure is directed, in some embodiments, to isolated nucleic acids, rAAVs, etc., comprising a codon-optimized nucleic acid sequence which encodes an SMN protein (e.g., SEQ ID NO: 4).

In some embodiments, the subject is a mammal. In some embodiments, the mammal is a human. In some embodiments, the subject has, is suspected of having, or is at risk of having, a neurological and/or neuromuscular disorder. In some embodiments, the subject has, is suspected of having, or is at risk of having, Spinal Muscular Atrophy (SMA). Other subjects are described elsewhere herein.

In some embodiments, the administration comprises systemic administration. In some embodiments, the systemic administration comprises intravenous injection. Other methods of administration are described elsewhere herein.

In some aspects, the disclosure relates to a method for treating SMA in a subject, the method comprising: administering to the subject an isolated nucleic acid, rAAV, or pharmaceutical composition as described herein.

As used herein, the term “preventing” refers to the application or administration of a composition comprising a transgene encoding an SMN protein to a subject, who has not had a symptom, or a disease associated with aberrant SMN activity but at a predisposition toward a disease associated with aberrant SMN activity, with the purpose to stop or slow down the development of one or more symptoms of the disease associated with aberrant SMN activity. In some embodiments, the disease is SMA. In some embodiments, “preventing” also refers to stop or slow down the development of a complication associated with aberrant SMN activity (e.g., SMA) as described herein.

As used herein, the term “treating” refers to the application or administration of a composition comprising a transgene encoding an SMN protein to a subject, who has a symptom or a disease associated with aberrant SMN activity, or a predisposition toward a disease associated with aberrant SMN activity, with the purpose to cure, heal, alleviate, relieve, alter, remedy, ameliorate, improve, or affect the disorder, the symptom of the disease, or the predisposition toward a disease associated with aberrant SMN activity. In some embodiments, the disease is SMA. In some embodiments, “treating” also refers to cure, heal, alleviate, relieve, alter, remedy, ameliorate, improve, or affect a complication associated with aberrant SMN activity (e.g., SMA) as described herein.

Alleviating a disease associated with aberrant SMN activity includes delaying the development or progression of the disease or reducing disease severity. Alleviating the disease does not necessarily require curative results. As used therein, “delaying” the development of a disease (such as a disease associated with aberrant SMN activity, such as SMA) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. Por purpose of this disclosure, development of progression refers to the biological course of the symptoms. “Development” includes occurrence, recurrence, and onset. As used herein “onset” or “occurrence” of a disease associated with aberrant SMN activity or angiogenesis includes initial onset and/or recurrence.

Compositions described herein may be administered (or a transgene expressed) in one or more target cells of a subject (e.g., a mammalian subject, such as a human). In some embodiments, a composition (e.g., isolated nucleic acid, rAAV, etc.) is administered to a cell of the central nervous system (CNS) of a subject. Examples of CNS cells include but are not limited to neurons, astrocytes, glial cells, etc.

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be a pharmaceutical formulation and dosage suitable for a particular application and for a method of administration of the agents. Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively, it may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to an animal, such as a syringe, topical application devices, or intravenous needle tubing and hag, particularly in the case of the kits for producing specific somatic animal models.

The kit may have a variety of forms, such as a blister pouch, a shrink-wrapped pouch, a vacuum scalable pouch, a scalable thermoformed tray, or a similar pouch or tray form, with the accessories loosely packed within the pouch, one or more tabes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for constructing an AAV vector as described herein. In addition, kits of the disclosure may include, instructions, a negative and/or positive control, containers, diluents and buffers for the sample, sample preparation tubes and a printed or electronic table of reference AAV sequence for sequence comparisons.

### EXAMPLES

### Example 1

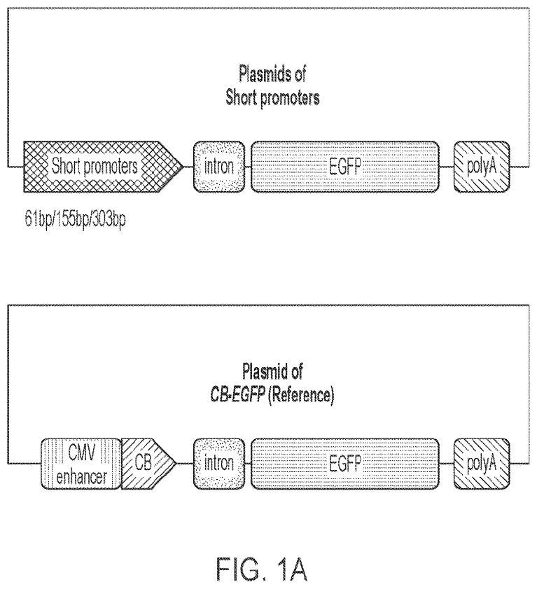

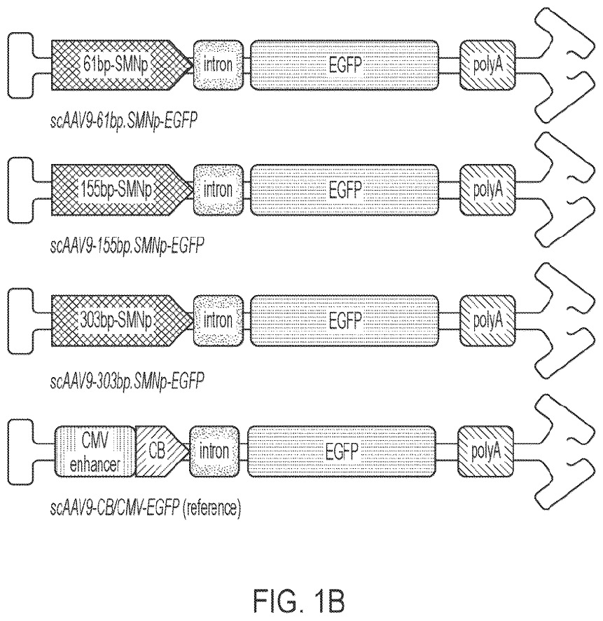

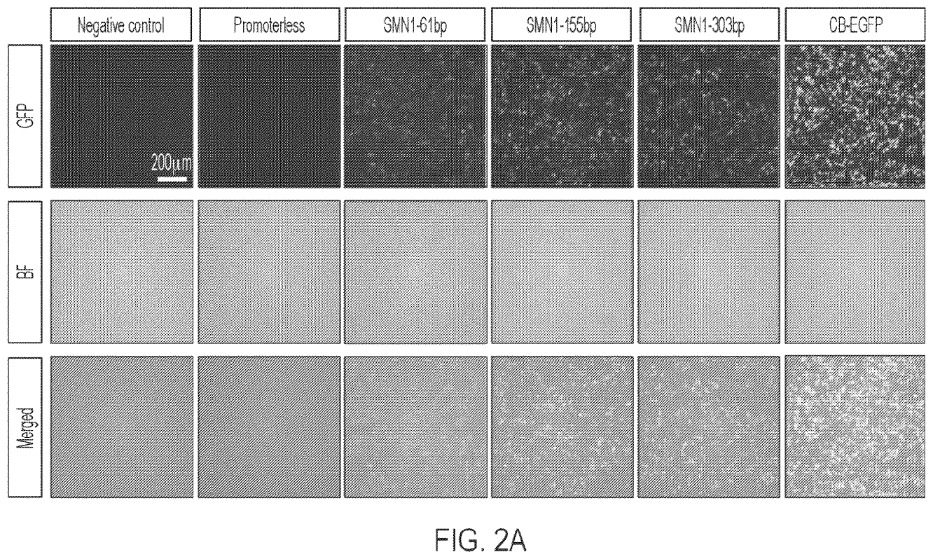

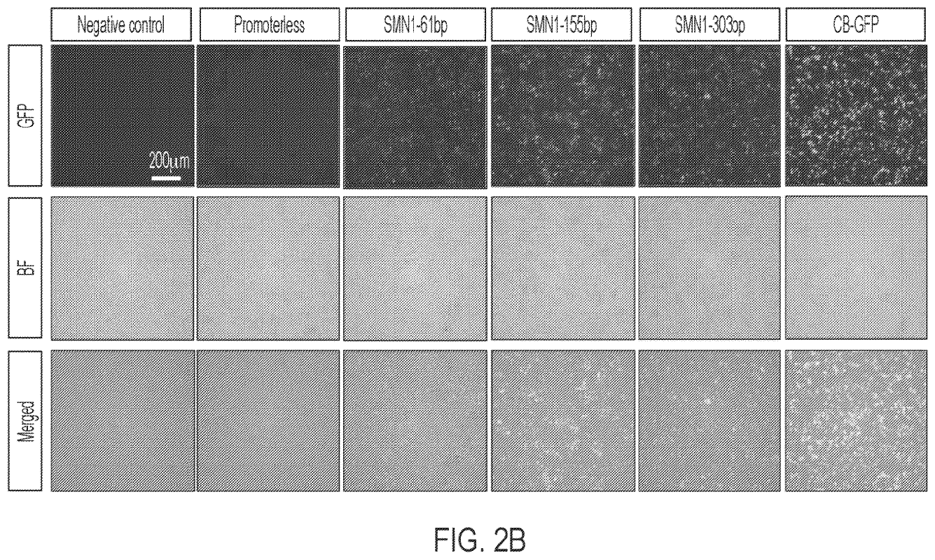

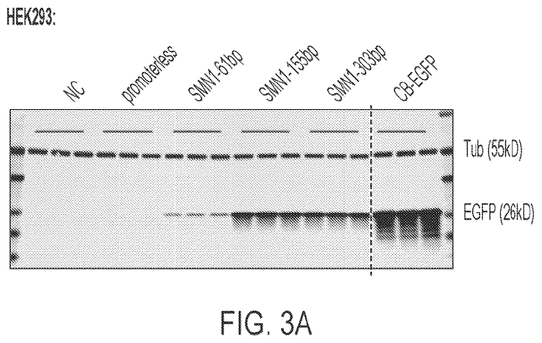

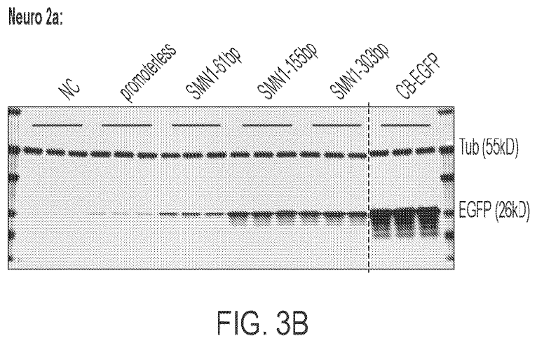

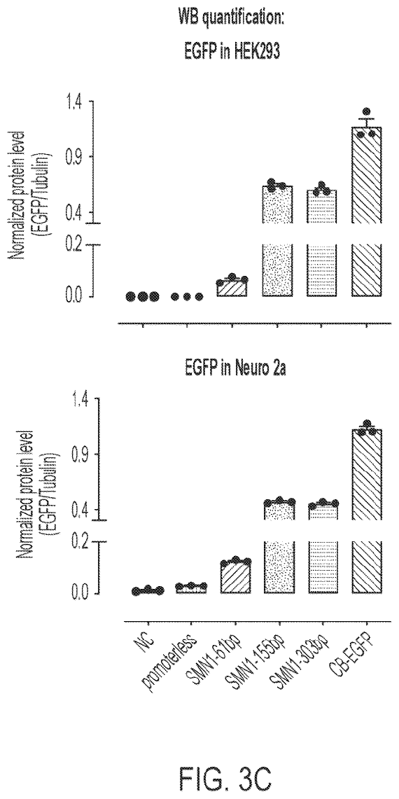

This example describes development of three compact promoters derived from the human survival motor neuron (SMN1) gene with lengths of 61 bp, 155 bp, and 303 bp. EGFP reporter plasmids harboring these three promoters were constructed and their activities in HEK293 and Neuro2a cells were evaluated by plasmid transfection (e.g., FIG. 1A). All three promoters drove efficient transgene expression in both cell lines, where the 155 bp promoter was the most efficient, and the 61 bp promoter was the least efficient (FIG. 2A-FIG. 3C), as measured by EGFP expression levels.

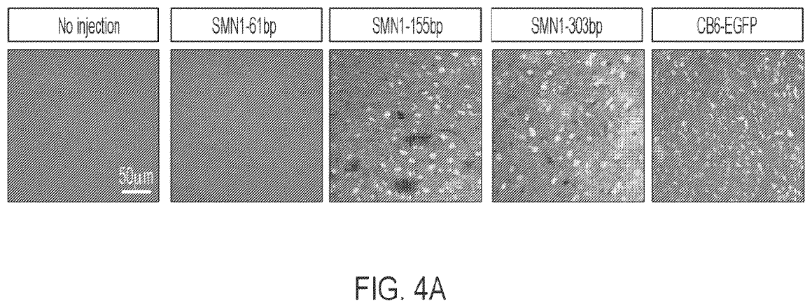

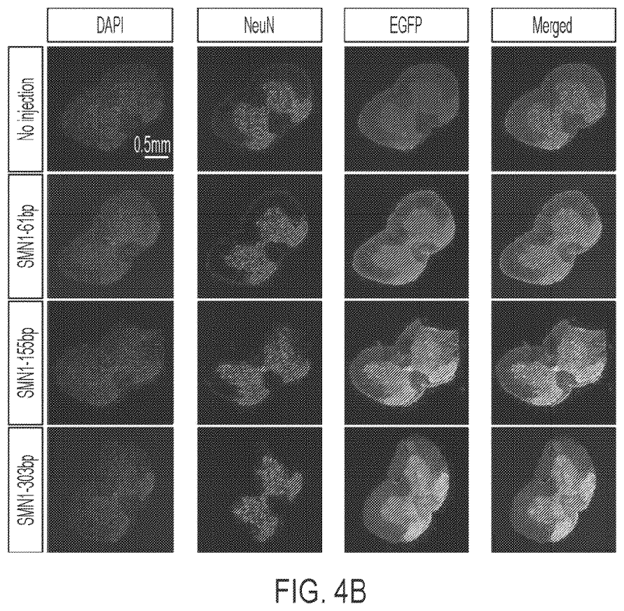

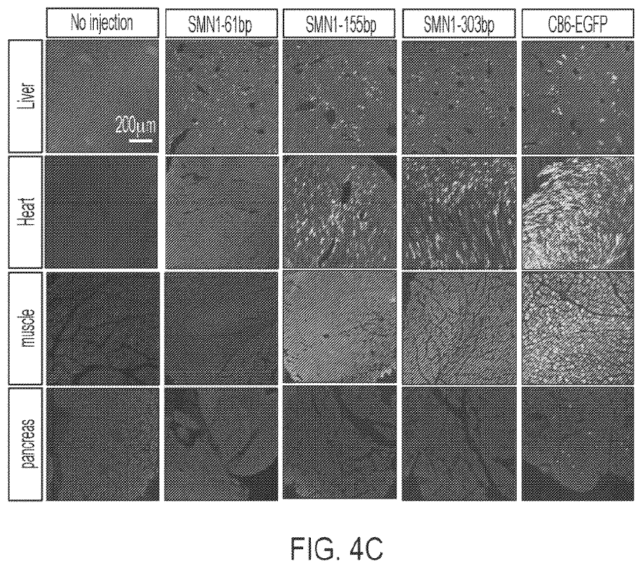

The reporter constructs were then packaged into self-complementary AAV9 (scAAV9) vectors and injected into neonatal FVB mice at P1 at a dose of 4×10¹¹ vg/mouse by facial vein administration. An scAAV9 vector with the cytomegalovirus enhancer/chicken β-actin (CMVen/CB) promoter driving an EGFP reporter (scAAV-CB/CMV-EGFP) was injected side-by-side for comparison (FIG. 1B). After three weeks, the mice were sacrificed and EGFP expression in brain, spinal cord, liver, heart, muscle, and pancreas was examined. Surprisingly, the 61-bp promoter exhibited spinal-cord-neuron- and heart-specific expression (FIG. 4B-FIG. 4C). However, no expression was detected in muscle (quadriceps) or pancreas, indicating that the 61-bp promoter was strongly active in neurons of the spinal cord (FIG. 4A-FIG. 4C). The 155-bp and 303-bp promoters showed comparable activity to the CB promoter in the brain and spinal cord (FIG. 4A-FIG. 4B). Interestingly, the 155-bp and 303-bp promoters also showed heart and muscle activity (FIG. 4C).

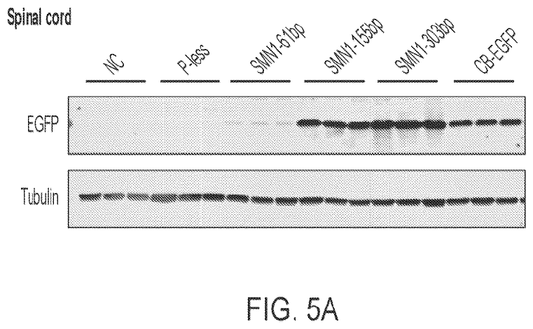

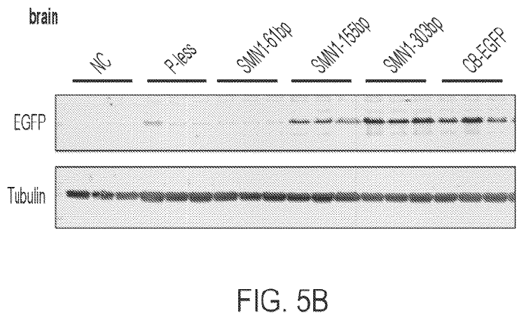

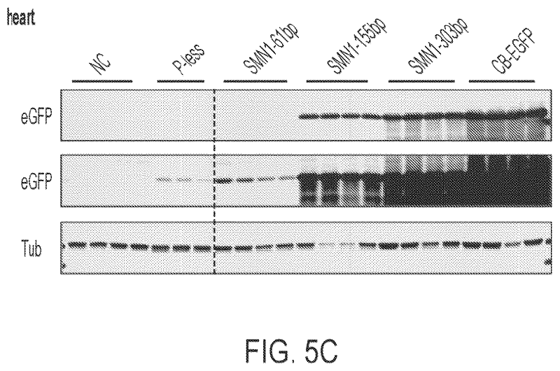

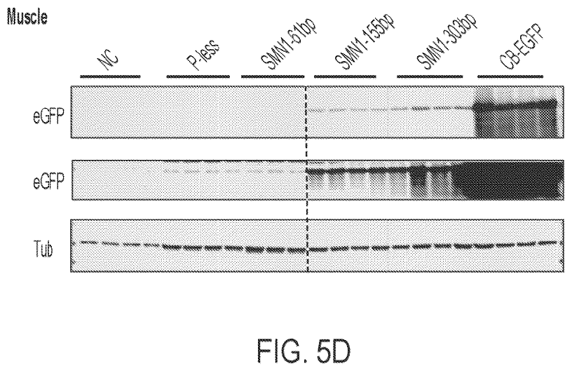

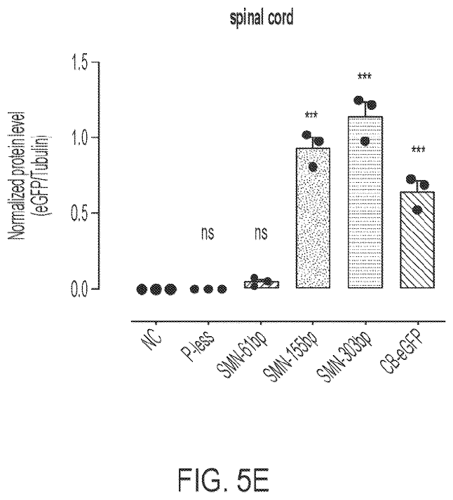

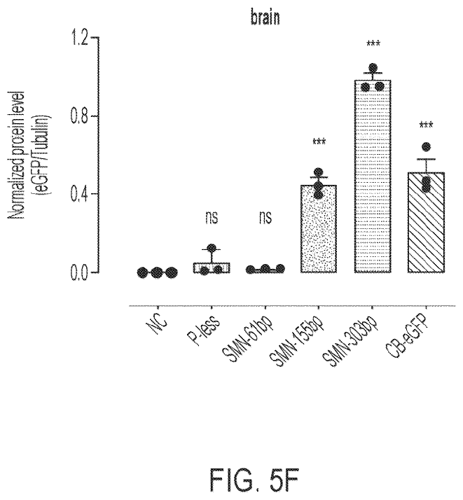

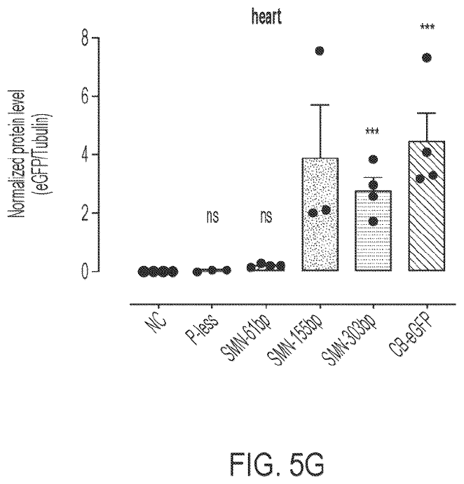

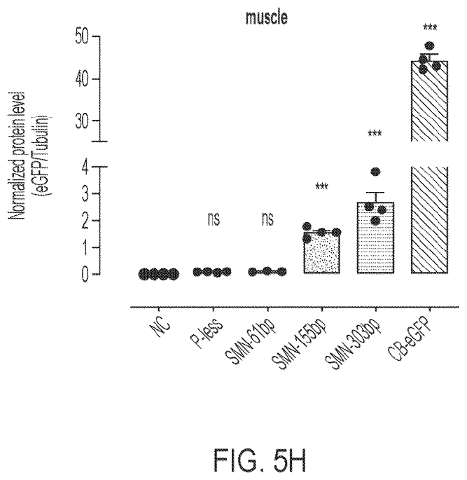

The EGFP level in different groups was also quantified (FIG. 5A-FIG. 5H), and the trend was consistent to the EGFP imaging.

This example has identified a small 61-bp promoter that is robustly active in, e.g., spinal cord neurons, and two additional small promoters with lengths of 155 bp and 303 bp with activities in, e.g., neurons and muscle cells.

### Example 2

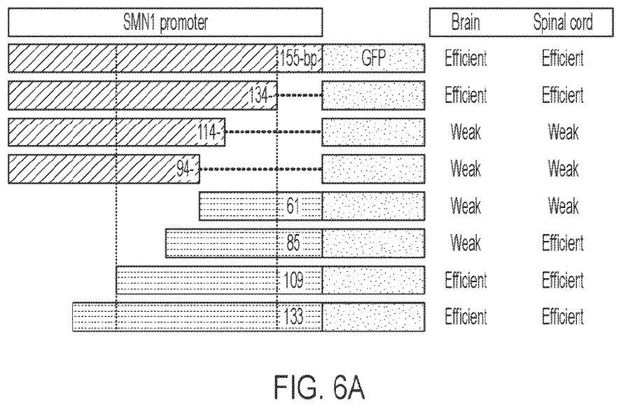

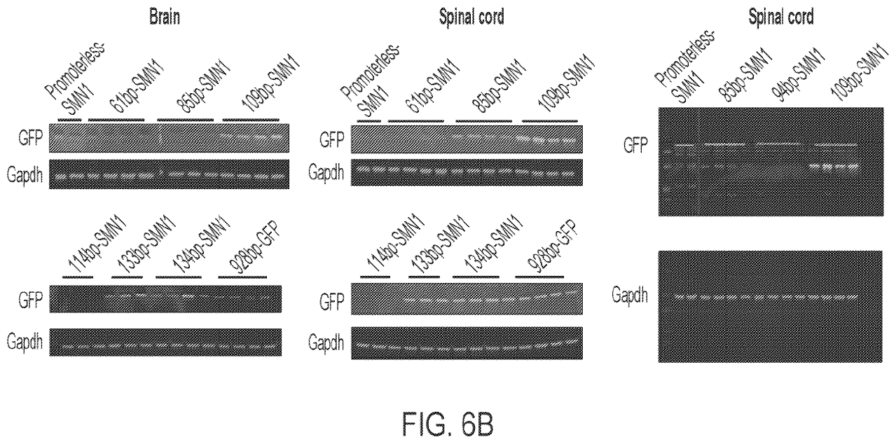

Six compact SMN1 promoters were generated by trimming a 155-bp SMN1 promoter from the 5 or 3′ end, then these AAV9-GFP reporter constructs driven by compact SMN1 promoters were packaged and neonatal B6 were injected at P1 by facial vein injection at the dose of 4×10¹¹ vg/mouse. After 4 weeks, animals were euthanized for analyzing the GFP expression levels in multiple tissues including brain and spinal cord. When the 155-bp promoter was trimmed from 3′ end, the 134-bp promoter, but not the 114-bp or 94-bp SMN1 promoter, showed efficient GFP expression in both brain and spinal cord. When the trimming started from the 5′ end, the 133- and 109-bp promoter showed efficient GFP expressions in both brain and spinal cord. The 85-bp promoter showed efficient GFP expression in spinal cord, but not in the brain (FIGS. 6A-6B).

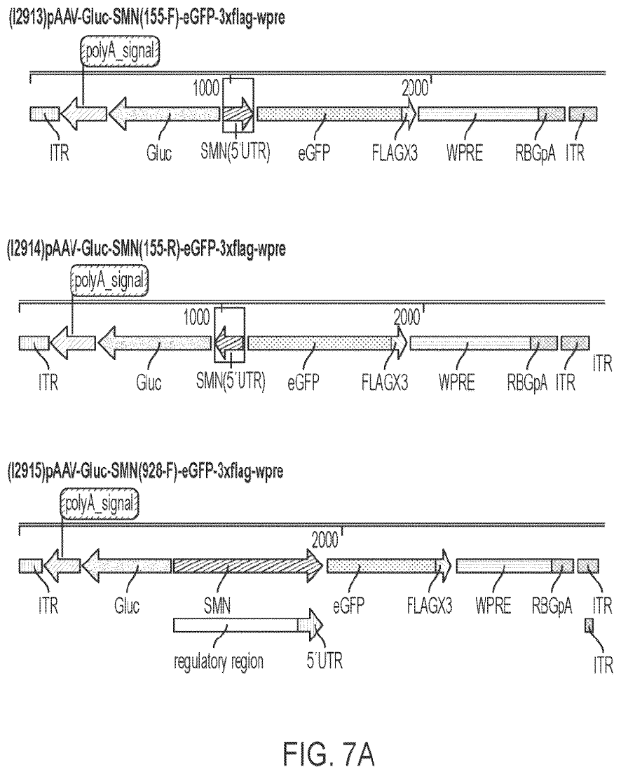

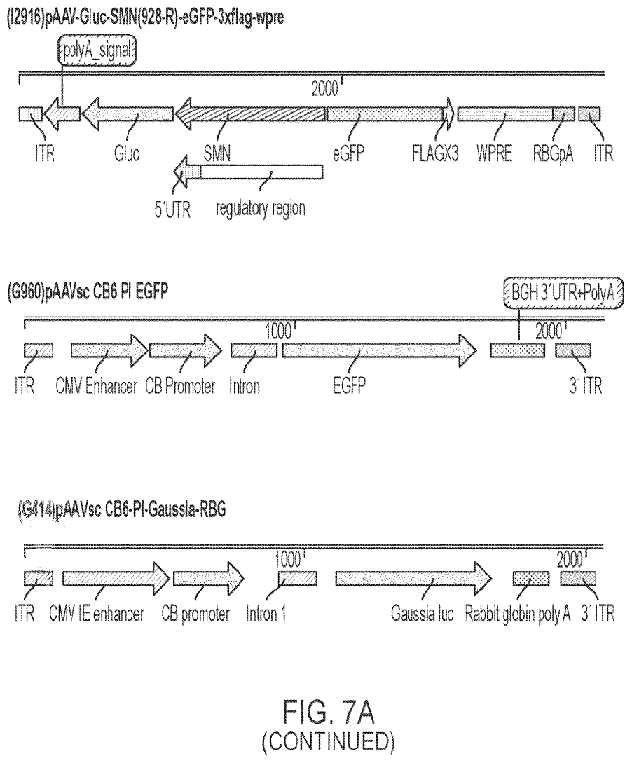

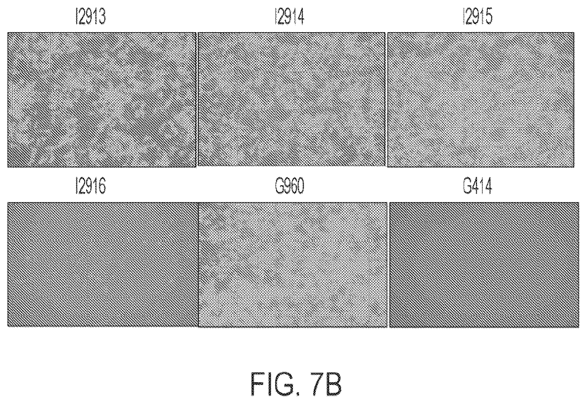

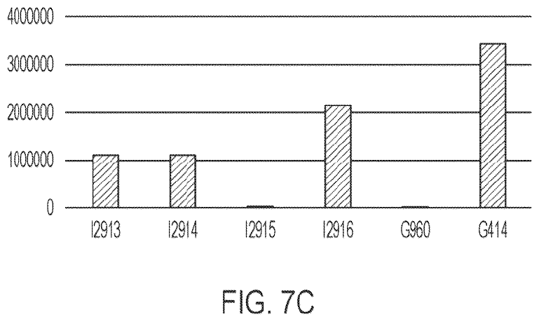

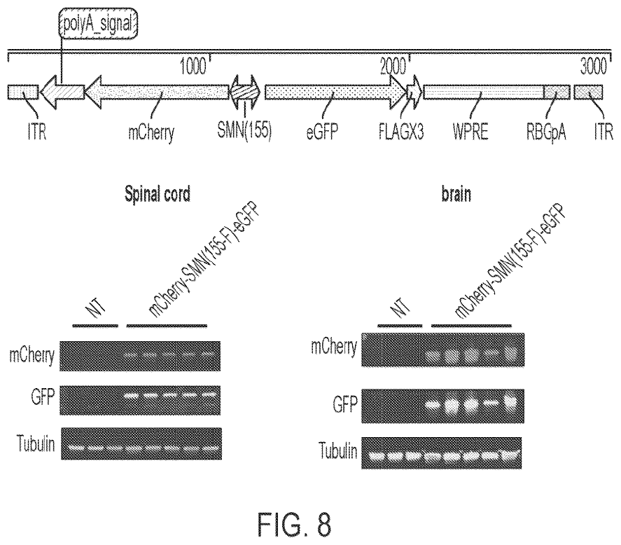

Meanwhile it was tested if the 155-bp SMN1 promoter could direct expression of two transgenes bidirectionally. To this end, AAV plasmids were constructed harboring the 155-bp SMN1 promoter in the middle flanking with the Gaussia luciferase gene and EGFP gene. The 155-bp SMN1 promoter was incorporated between two reporter genes at two orientations (SMN-155F and SMN1-155R) by plasmid transfection into HEK293 cells. The 155-bp promoter was capable of efficiently driving both Gaussia luciferase and EGFP expression at comparable levels. In contrast, a longer 928-bp SMN1 promoter could only drive its downstream transgene expression (FIG. 7A-7C).

A dual reporter construct harboring GFP and mCherry was packaged into rAAV9 for facial vein administration to neonatal C57BL/6 mice at P1 with a dose of 4×10¹¹ vg/mouse. Four weeks after injection, it was found that the 155 bp promoter drove both GPP and mCherry expression in the brain and spinal cord.

REPRESENTATIVE SEQUENCES

> SMN1 promoter (61 bp; SEQ ID NO: 1):
gccggaagtcgtcactcttaagaagggacggggccccacgctgcgcaccc
gcgggtttgct
> SMN1 promoter (155 bp; SEQ ID NO: 2);
gtgggagggcgataaccactcgtagaaagcgtgagaagttactacaagcg
gtcctcccgggcaccgtactgttccgctcccagaagccccgggcgcggaa
gtcgtcactcttaagaagggacggggccccacgctgcgcacccgcgggtt
tgct
> SMN1 promoter (303 bp; SEQ ID NO: 3):
Gtgagccgagatcgcgccgctgcactccagcctgagcgacagggcgaggc
tctgtctcaaaacaaacaaacaaaaaaaaaaaggaaaggaaatataacac
agtgaaatgaaaggattgagagaaatgaaaaatatacacgccacaaatgt
gggagggcgataaccactcgtagtaaagcgtgagaagttactacaagcgg
tcctcccgggcaccgtactgttccgctcccagaagccccgggcgccggaa
gtcgtcactcttaagaagggacggggccccacgctgcgcacccgcgggtt
tgct
> Codon-optimized human SMN1 nucleic acid
sequence (SEQ ID NO: 4)
ATGGCCATGAGCAGCGGCGGCAGTGGCGGCGGCGTGCCCGAGCAGGAGGA
TTCTGTGCTGTTCCGGAGAGGAACAGGCCAGAGCGATGACTCCGATATCT
GGGACGACACAGCCCTTATCAAGGCCTACGACAAGGCCGTGGCCAGCTTT
AAGCACGCCCTGAAGAATGGCGATATCTGCGAGACAAGCGGAAAGCCTAA
GACCACOCCTAAAAGAAAGCCCGCCAAGAAAAACAAGTCCCAGAAAAAAA
ACACCGCCGCTAGCCTGCAGCAGTGGAAGGTGGGCGACAAATGCAGCGCC
ATCTGGTCCGAGGACGGCTGCATCTACCCTGCTACCATCGCCAGCATCGA
CTTCAAGCGGGAAACCTGCGTGGTGGTCTACACAGGCTATGGCAATAGGG
AGGAACAAAATCTCTCTGATCTGCTGTCTCCTATTTGTGAAGTGGCTAAC
AACATCGAGCAGAACGCCCAGGAAAATGAGAACGAAAGCCAAGTGTCCAC
CGACGAGAGCGAGAACAGCAGAAGCCCTGGAAACAAGTCTGACAACATCA
AGCCCAAGTCTGCCCCTTGGAACAGCTTCCTGCCCCCTCCTCCTCCAATG
CCTGGCCCCAGACTGGGCCCCGGCAAGCCTGGCCTGAAGTTCAACGGCCC
TCCTCCACCCCCTCCTCCTCCACCTCCCCATCTGCTGAGCTGCTGGCTGC
CTCCTTTTCCCAGCGGCCCCCCTATCATCCCCCCACCACCTCCTATCTGT
CCCGACAGCCTGGACGACGCCGATGCTCTGGGATCCATGCTGATCAGCTG
GTACATGTCTGGCTACCACACCGGCTACTACATGGGCTTCCGGCAGAACC
AGAAGGAAGGAAGATOCAGCCACAGCCTGAACTGA
> SMN1 promoter nucleic acid sequence 1 (SEQ ID
NO: 5)
tcgaagctttataaaaacatacttttttttttacttttttttttttttct
gagacacagcctcactctgtcgcccaggctggagtgcaggttttcatgtt
tatctgtgagatgtacctttggcacattactttcctgacatgagatttaa
atttttttttttatcttgtgacaatttaacttttttgacacataaaaaat
tgtacatatttatttgtttgagatggagtcgcactctgcactcaggctgg
agtgcagtggcgtgatcttggctcactgcaacctccgcctcccgagttca
agtgattctcctggctcagcctcccaagcagctgtcattacaggcctgca
ccaccacacccggctgatttttgtatttttaggagaaacagggtttcacc
atgttgggccaggctggtcttgaagtcctgacctcaagtgatccacccac
cttggcctcccaaagtgctgggattataggcatgagccaccgtaccagac
ccctaaaaattgtatatatttaaggtgtaccatttgatgtttagatatac
attgtgaaatgattacattccacatattacctctacagagttaccatttt
tgtacacttggtcaacatcatcccattctccccttcctccacagatattt
cttgtatactatatagaagccaagggtattttgggggaagagctcaaagt
tcctttcgtggagttaaaaatatatatatactatgtacatataagccatt
tagcaaccctagatgcttaataaagaatactggaggcccggtgtggtggc
tcacacctgtaatcccagcactttgggaggccgaggcggtcggattacga
ggtcaggagttcaagaccagcctggccaacatggtgaaaccccatcttta
ctaaaaatacaaaaattagccgggtgtggtggtgggcgcctgtaatccca
gctactcggggggctgaggcagaattgcttgaacctgggaggcagaggtt
gcagtgagctgagatcacgccactgcattccagcctgggtgacagagcaa
tattctgtcgcaaaaaaaaaaagaatactggaggctgggcgaggtggctc
acacctgtaatcccagcattttgggatgccagaggcgggcggaatntctt
gagctcaggagttcgagaccagcctacacaatatgctccaaacgccgctt
ntacaaaacatacagaaactacccgggtgtggtggcgnncccctgtggtc
ctagatacttgggaggttgaggcgggaggatcgcttgagctcgggaggtc
gaggctgcaatgagccgagatggtgccactgcattctgacgacagagcga
gattccgtttcaaaacaaacaacaaataaggttgggggatcaaatatctt
ctagtgtttaaggatctgccttccttcctgcccccatgtttgtctttcct
tgtttgtctttatatagatcaagcaggttttaaattcctagtaggagctt
acatttacttttccaagggggagggggaataaatatctacacacacacac
acacacacacacacacacacacacacacacacacacacacaccacactgg
agttcgagacgaggcctaagcaacatgccgaaaccccgtctctactaaat
acaaaaaatagctgagcttggtggcgcacgcctatagtcctagctactgg
ggaggctgaggtgggaggatcgcttgagcccaagaagtcgaggctgcagt
gagccgagatcgcgccgctgcactccagcctgagcgacagggcgaggctc
tgtctcaaaacaaacaaacaaaaaaaaaaaggaaaggaaatataacacag
tgaaatgaaaggattgagagaaatgaaaaatatacacgccacaaatgtgg
gagggcgataaccactcgtagaaagcgtgagaagttactacaagcggtcc
tcccgggcaccgtactgttccgctcccagaagccccgggcgccggaagtc
gtcactcttaagaagggacggggccccacgctgcgcacccgcgggtttgc
t
> SMN1 promoter nucleic acid sequence 2 (SEQ ID
NO: 6)
ggatgccagaggcgggcggaatntcttgagctcaggagttcgagaccagc
ctacacaatatgctccaaacgccgcttntacaaaacatacagaaactacc
cgggtgtggtggcgnncccctgtggtcctagatacttgggaggttgaggc
gggaggatcgcttgagctcgggaggtcgaggctgcaatgagccgagatgg
tgccactgcattctgacgacagagcgagattccgtttcaaaacaaacaac
aaataaggttgggggatcaaatatcttctagtgtttaaggatctgccttc
cttcctgcccccatgtttgtctttccttgtttgtctttatatagatcaag
caggttttaaattcctagtaggagcttacatttacttttccaagggggag
ggggaataaatatctacacacacacacacacacacacacacacacacaca
cacacacacacacacacaccacactggagttcgagacgaggcctaagcaa
catgccgaaaccccgtctctactaaatacaaaaaatagctgagcttggtg
gcgcacgcctatagtcctagctactggggaggctgaggtgggaggatcgc
ttgagcccaagaagtcgaggctgcagtgagccgagatcgcgccgctgcac
tccagcctgagcgacagggcgaggctctgtctcaaaacaaacaaacaaaa
aaaaaaaggaaaggaaatataacacagtgaaatgaaaggattgagagaaa
tgaaaaatatacacgccacaaatgtgggagggcgataaccactcgtagaa
agcgtgagaagttactacaagcggtcctcccgggcaccgtactgttccgc
tcccagaagccccgggcgccggaagtcgtcactcttaagaagggacgggg
ccccacgctgcgcacccgcgggtttgct

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one. B, with no A present (and optionally including elements other than A); in yet another embodiment, to at least one, optionally including more than one, A, and at least one, optionally including more than one. B (and optionally including other elements); etc.

In the claims, as well as in the specification above, all transitional phrases such as “comprising,” “including.” “carrying,” “having.” “containing.” “involving.” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal terra) to distinguish the claim elements.

1. An isolated nucleic acid comprising a human SMN1 promoter variant having a nucleic acid sequence that is at least 60% identical to the nucleic acid sequence set forth in any one of SEQ ID NOs: 1-3 and does not comprise the sequence set forth in SEQ ID NO: 5 or 6.

2. The isolated nucleic acid of claim 1, wherein the human SMN1 promoter variant comprises or consists of the nucleic acid sequence set forth in SEQ ID NO: 1.

3. The isolated nucleic acid of claim 1, wherein the human SMN1 promoter variant comprises or consists of the nucleic acid sequence set forth in SEQ ID NO: 2.

4. The isolated nucleic acid of claim 1, wherein the human SMN1 promoter variant comprises or consists of the nucleic acid sequence set forth in SEQ ID NO: 3.

5. The isolated nucleic acid of claim 1, further comprising a nucleic acid sequence encoding one or more gene products operably linked to the human SMN1 promoter variant.

6. The isolated nucleic acid of claim 5, wherein the gene product is a protein or an interfering nucleic acid.

7. The isolated nucleic acid of claim 6, wherein the interfering nucleic acid is a dsRNA, siRNA, shRNA, miRNA, artificial miRNA (ami-RNA), or RNA aptamer.

8. The isolated nucleic acid of claim 5, wherein the gene product is a therapeutic protein, optionally wherein the therapeutic protein is a SMN1 protein.

9. The isolated nucleic acid of claim 1, further comprising a polyA region.

10. The isolated nucleic acid of claim 1, further comprising one or more adeno-associated virus (AAV) inverted terminal repeats (ITRs), optionally wherein the AAV ITRs are AAV2 ITRs.

11. A vector comprising the isolated nucleic acid of claim 1.

12. The vector of claim 11, wherein the vector is a plasmid.

13. A recombinant AAV (rAAV) comprising:
(i) the isolated nucleic acid of claim 1; and
(ii) at least one AAV capsid protein.

14. The rAAV of claim 13, wherein the at least one capsid protein has a serotype selected from an AAV2, AAV3, AAV4, AAV5, AAV6, AAV7, AAV8, AAVrh8, AAV9, AAV10, or AAVrh10.

15. The rAAV of claim 13, wherein the at least one capsid protein is an AAV9 capsid protein.

16. The rAAV of claim 13, wherein the rAAV is a self-complementary AAV (scAAV).

17. The rAAV of claim 13, wherein the isolated nucleic acid encodes a Survival Motor Neuron (SMN) protein.

18. A method of expressing a gene product in a subject, the method comprising administering the isolated nucleic acid of claim 1 to the subject.

19. The method of claim 18, wherein the subject is a mammal.

20. The method of claim 19, wherein the mammal is a human.

21-25. (canceled)
